(19) United States
(12) Patent Application Publication
(10) Pub. No.: US 2024/0190806 A1
(43) Pub. Date: Jun. 13, 2024

(54) CHEMICAL PROCESS

(71) Applicant: SYNGENTA CROP PROTECTION AG, Basel (CH)

(72) Inventors: Roman STAIGER, Stein (CH); Renaud BEAUDEGNIES, Stein (CH)

(73) Assignee: SYNGENTA CROP PROTECTION AG, Basel (CH)

(21) Appl. No.: 18/552,616

(22) PCT Filed: Mar. 25, 2022
(86) PCT No.: PCT/EP2022/057941

§ 371 Date: Sep. 26, 2023

(30) Foreign Application Priority Data
Mar. 26, 2021 (GB) 2104286.6

(51) Int. Cl. C07C 69/736

(52) U.S. Cl. CPC C07C 69/736; C07C 2601/08; C07C 2601/14; C07C 2601/16

(57) ABSTRACT

The present invention provides, inter alia, a process for producing a compound of formula (I) wherein the substituents are as defined in claim 1. The present invention further provides intermediate compounds utilised in said process, and methods for producing said intermediate compounds.

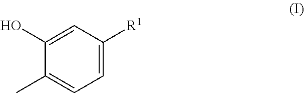

The present invention relates to a novel process for the synthesis of certain cycloalkyl substituted phenol compounds. Such compounds are useful intermediates in the synthesis of microbiocidal methoxyacrylate compounds, which have microbiocidal activity, in particular, fungicidal activity. Such compounds are known, for example, from WO 2020/193387 and processes for making such compounds or intermediates thereof are also known. Such compounds are typically produced via a hydrogenation of a cycloalkene intermediate or a cross-coupling reaction between a halo substituted intermediate and an organometallic or organometalloid species in the presence of a suitable catalyst.

The hydrogenation of a cycloalkene intermediate is known (see for example WO 2020/193387), however, such a process has a number of drawbacks. Firstly, this approach often leads to lengthy reaction times and secondly, requires an increased number of steps to obtain the desired fungicidal methoxyacrylate compounds. The cross-coupling approach also has a number of drawbacks, in that it typically involves the use of expensive catalysts and generates undesirable by-products. Thus, such approaches are not ideal for large scale production and therefore a new, more efficient synthesis method is desired to avoid the generation of undesirable by-products.

The present invention provides a Friedel-Crafts alkylation process which (i) avoids the need for a hydrogenation and (ii) avoids the need for a halo substituted phenyl derivative. The Friedel-Crafts alkylation of ortho-cresol with isopropyl chloride has been described (U.S. Pat. No. 2,064,885), however, the reaction produces a mixture of isomeric products. Surprisingly, we have now found that a selective mono-alkylation to deliver the desired meta isomer, a compound of formula (I), can be achieved in the process of the present invention which in turn can be converted to the desired fungicidal methoxyacrylate compounds. Such a process is more convergent and atom efficient, which may be more cost effective and produce less waste products.

Thus, according to the present invention there is provided a process for the preparation of a compound of formula (I) or a salt thereof:

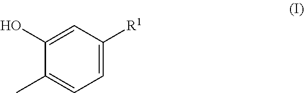

wherein
R¹ is C₃-C₇cycloalkyl;
said process comprising:
reacting a compound of formula (II)

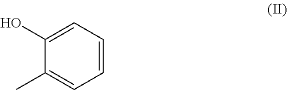

with a compound of formula (Ill)

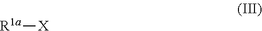

wherein R^{1a} is C₃-C₇cycloalkyl and X is halogen or hydroxy; or
R^{1a} is C₃-C₇cycloalkenyl and X is hydrogen;
in the presence of an acid to give a compound of formula (I).

According to a second aspect of the invention, there is provided an intermediate compound of formula (V),

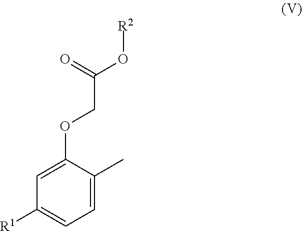

wherein the intermediate compound of formula (V) is selected from the group consisting of a compound of formula (V-I), (V-II), (V-III) and (V-IV) below,

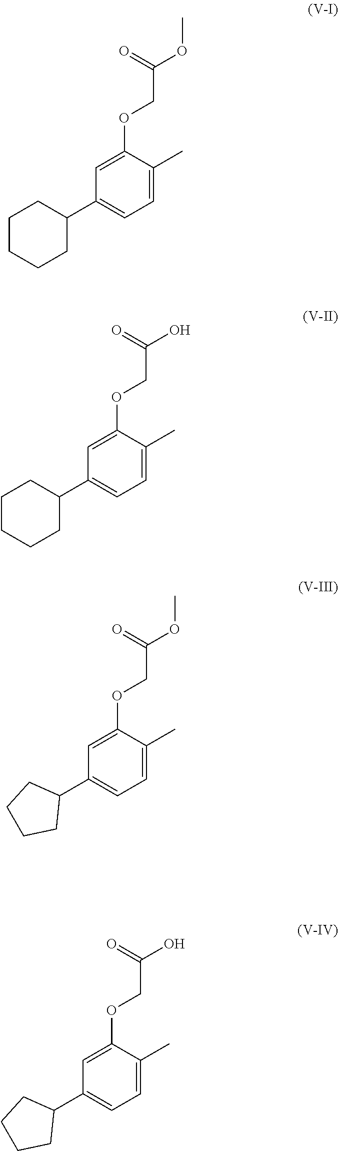 [SMILES: COC(=O)COc1cc(C2CCCCC2)ccc1C ; Cc1ccc(C2CCCCC2)cc1OCC(=O)O ; COC(=O)COc1cc(C2CCCC2)ccc1C ; Cc1ccc(C2CCCC2)cc1OCC(=O)O]

According to a third aspect of the invention, there is provided the use of a compound of formula (I),

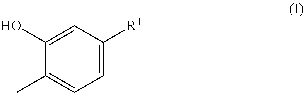

wherein R¹ is as defined herein, for preparing a compound of formula (VI),

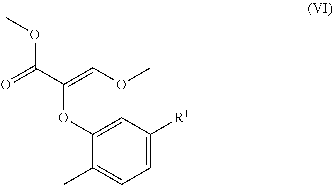

wherein R¹ is as defined herein.

As used herein, the term “halogen” refers to fluorine (fluoro), chlorine (chloro), bromine (bromo) or iodine (iodo).

As used herein, the term “hydroxyl” or “hydroxy” means an —OH group.

As used herein, the term “C₁-C₆alkyl” refers to a straight or branched hydrocarbon chain radical consisting solely of carbon and hydrogen atoms, containing no unsaturation, having from one to six carbon atoms, and which is attached to the rest of the molecule by a single bond. C₁-C₄alkyl and C₁-C₂alkyl are to be construed accordingly. Examples of C₁-C₆alkyl include, but are not limited to, methyl, ethyl, n-propyl, 1-methylethyl (iso-propyl), n-butyl, and 1-dimethylethyl (t-butyl).

As used herein, the term “C₃-C₇cycloalkyl” refers to a stable, monocyclic ring radical which is saturated and contains 3 to 7 carbon atoms. Examples of C₃-C₇cycloalkyl include, but are not limited to, cyclopropyl, cyclobutyl, cyclopentyl and cyclohexyl.

As used herein, the term “C₃-C₇cycloalkenyl” refers to a radical which is a monocyclic non-aromatic ring system consisting solely of carbon and hydrogen atoms and which contains 3 to 7 carbon atoms and 1 endocyclic double bond. Examples of C₃-C₇cycloalkenyl include, but are not limited to, cyclobutenyl, cyclopentenyl, cyclohexenyl and cycloheptenyl.

The process of the present invention can be carried out in separate process steps, wherein the intermediate compounds can be isolated at each stage. Alternatively, the process can be carried out in a one-step procedure wherein the intermediate compounds produced are not isolated. Thus, it is possible for the process of the present invention to be conducted in a batch wise or continuous fashion.

The compounds of formula (I) could equally be represented in unprotonated or salt form with one or more relevant counter ions. This invention covers processes to make all such salts and mixtures thereof in all proportions. For example a compound of formula (I) may exist as a salt, a compound of formula (I-I) wherein M represents a suitable cation and R¹ is as defined herein,

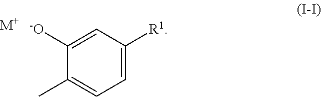

Suitable cations represented by M include, but are not limited to, metals, conjugate acids of amines and organic cations. Examples of suitable metals include aluminium, calcium, cesium, copper, lithium, magnesium, manganese, potassium, sodium, iron and zinc. Examples of suitable amines include allylamine, ammonia, amylamine, arginine, benethamine, benzathine, butenyl-2-amine, butylamine, butylethanolamine, cyclohexylamine, decylamine, diamylamine, dibutylamine, diethanolamine, diethylamine, diethylenetriamine, diheptylamine, dihexylamine, diisoamylamine, diisopropylamine, dimethylamine, dioctylamine, dipropanolamine, dipropargylamine, dipropylamine, dodecylamine, ethanolamine, ethylamine, ethylbutylamine, ethylenediamine, ethylheptylamine, ethyloctylamine, ethylpropanolamine, heptadecylamine, heptylamine, hexadecylamine, hexenyl-2-amine, hexylamine, hexylheptylamine, hexyloctylamine, histidine, indoline, isoamylamine, isobutanolamine, isobutylamine, isopropanolamine, isopropylamine, lysine, meglumine, methoxyethylamine, methylamine, methylbutylamine, methylethylamine, methylhexylamine, methylisopropylamine, methylnonylamine, methyloctadecylamine, methylpentadecylamine, morpholine, 1,4-diazabicyclo[2.2.2]octane, 1,8-diazabicyclo[5.4.0]undec-7-ene, 1,5-diazabicyclo[4.3.0]non-5-ene, quinuclidine, N-methylpyrrolidine, N,N-diethylethanolamine, N-methylpiperazine, nonylamine, octadecylamine, octylamine, oleylamine, pentadecylamine, pentenyl-2-amine, phenoxyethylamine, picoline, piperazine, piperidine, propanolamine, propylamine, propylenediamine, pyridine, pyrrolidine, sec-butylamine, stearylamine, tallowamine, tetradecylamine, tributylamine, tridecylamine, trimethylamine, triheptylamine, trihexylamine, triisobutylamine, triisodecylamine, triisopropylamine, trimethylamine, tripentylamine, tripropylamine, tris(hydroxymethyl)aminomethane, and undecylamine. Examples of suitable organic cations include benzyltributylammonium, benzyltrimethylammonium, benzyltriphenylphosphonium, choline, tetrabutylammonium, tetrabutylphosphonium, tetraethylammonium, tetraethylphosphonium, tetramethylammonium, tetramethylphosphonium, tetrapropylammonium, tetrapropylphosphonium, tributylsulfonium, tributylsulfoxonium, triethylsulfonium, triethylsulfoxonium, trimethylsulfonium, trimethylsulfoxonium, tripropylsulfonium and tripropylsulfoxonium. Emphasis is given to calcium, cesium, lithium, magnesium, potassium, sodium and zinc salts.

The following list provides definitions, including preferred definitions, for substituents X, Y, R¹, R^{1a} and R² with reference to the process according to the invention. For any one of these substituents, any of the definitions given below may be combined with any definition of any other substituent given below or elsewhere in this document.

R¹ is C₃-C₇cycloalkyl. Preferably, R¹ is selected from the group consisting of cyclopropyl, cyclobutyl, cyclopentyl and cyclohexyl. More preferably, R¹ is selected from the group consisting of cyclopropyl, cyclopentyl and cyclohexyl. Even more preferably, R¹ is cyclopentyl or cyclohexyl. Most preferably, R¹ is cyclohexyl.

R^{1a} is C₃-C₇cycloalkyl and X is halogen or hydroxy. Preferably, R^{1a} is selected from the group consisting of cyclopropyl, cyclobutyl, cyclopentyl and cyclohexyl and X is halogen or hydroxy. More preferably, R^{1a} is selected from the group consisting of cyclopropyl, cyclopentyl and cyclohexyl and X is halogen or hydroxy. Even more preferably, R^{1a} is cyclopentyl or cyclohexyl and X is halogen or hydroxy. Even more preferably still, R^{1a} is cyclopentyl or cyclohexyl and X is selected from the group consisting of chloro, bromo and hydroxy. Yet even more preferably still, R^{1a} is cyclopentyl or cyclohexyl and X is chloro or hydroxy. Furthermore preferably still, R^{1a} is cyclohexyl and X is chloro or hydroxy (preferably, X is chloro).

Alternatively, R^{1a} is C₃-C₇cycloalkenyl and X is hydrogen. Preferably, R^{1a} is selected from the group consisting of cyclopropenyl, cyclobutenyl, cyclopentenyl and cyclohexenyl and X is hydrogen. More preferably, R^{1a} is selected from the group consisting of cyclopropenyl, cyclopentenyl and cyclohexenyl and X is hydrogen. Even more preferably, R^{1a} is cyclopentenyl or cyclohexenyl and X is hydrogen. Most preferably, R^{1a} is cyclohexenyl and X is hydrogen.

R² is selected from the group consisting of hydrogen and C₁-C₆alkyl. Preferably, R² is selected from the group consisting of hydrogen, methyl and ethyl. More preferably, R² is hydrogen or methyl. Most preferably, R² is methyl.

In one embodiment R² is hydrogen.

In one embodiment of the invention the compound of formula (III) is selected from the group consisting of chlorocyclopentane, bromocyclopentane, chlorocyclohexane, bromocyclohexane, cyclopentanol, cyclohexanol, cyclopentene and cyclohexene. Preferably, the compound of formula (III) is selected from the group consisting of chlorocyclopentane, chlorocyclohexane, cyclopentanol, cyclohexanol, cyclopentene and cyclohexene. More preferably, the compound of formula (III) is selected from the group consisting of chlorocyclohexane, cyclohexanol and cyclohexene. Even more preferably, the compound of formula (III) is chlorocyclohexane or cyclohexanol. Most preferably, the compound of formula (III) is chlorocyclohexane.

Y is a suitable leaving group (such as a halogen or sulfonate). Preferably, Y is selected from the group consisting of halogen, CF₃S(O)₂O—, (p-tolyl)S(O)₂O— and CH₃S(O)₂O—. More preferably, Y is halogen. Even more preferably, Y is chloro or bromo. Most preferably, Y is chloro.

The present invention further provides an intermediate compound of formula (V)

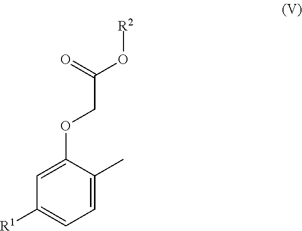

wherein R¹ and R² are as defined herein.

Preferably, in an intermediate compound of formula (V), R¹ is selected from the group consisting of cyclopropyl, cyclobutyl, cyclopentyl and cyclohexyl and R² is hydrogen or methyl. More preferably, R¹ is selected from the group consisting of cyclopropyl, cyclopentyl and cyclohexyl and R² is hydrogen or methyl.

Even more preferably, the intermediate compound of formula (V) is selected from the group consisting of a compound of formula (V-I), (V-II), (V-III) and (V-IV) below,

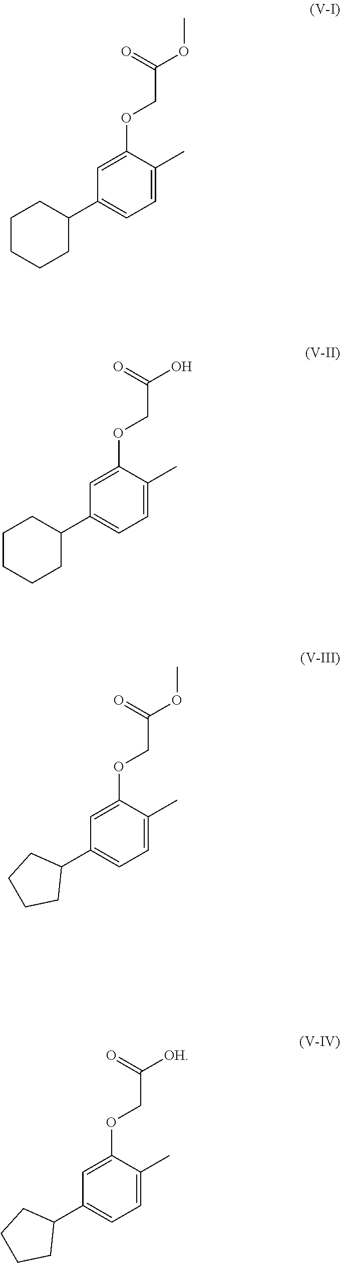 [SMILES: COC(=O)COc1cc(C2CCCCC2)ccc1C ; Cc1ccc(C2CCCCC2)cc1OCC(=O)O ; COC(=O)COc1cc(C2CCCC2)ccc1C ; Cc1ccc(C2CCCC2)cc1OCC(=O)O]

Even more preferably still, the intermediate compound of formula (V) is a compound of formula (V-I) or (V-II). Most preferably, the intermediate compound of formula (V) is a compound of formula (V-I).

In one embodiment, the intermediate compound of formula (V) is a compound of formula (V-II).

The present invention further provides an intermediate compound of formula (VII)

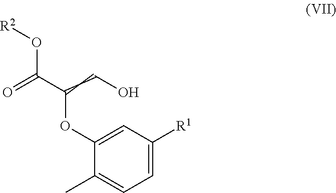

wherein R¹ and R² are as defined herein.

Preferably, in an intermediate compound of formula (VII), R¹ is selected from the group consisting of cyclopropyl, cyclobutyl, cyclopentyl and cyclohexyl and R² is hydrogen or methyl. More preferably, R¹ is selected from the group consisting of cyclopropyl, cyclopentyl and cyclohexyl and R² is hydrogen or methyl.

Even more preferably, the intermediate compound of formula (VII) is selected from the group consisting of a compound of formula (VII-I), (VII-II), (VII-III) and (VII-IV) below,

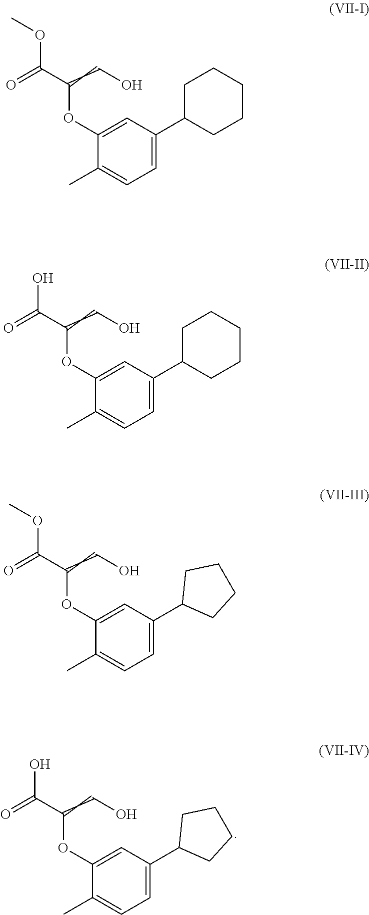 [SMILES: COC(=O)C(=CO)Oc1cc(C2CCCCC2)ccc1C ; Cc1ccc(C2CCCCC2)cc1OC(=CO)C(=O)O ; COC(=O)C(=CO)Oc1cc(C2CCCC2)ccc1C ; Cc1ccc(C2CCCC2)cc1OC(=CO)C(=O)O]

Even more preferably still, the intermediate compound of formula (VII) is a compound of formula (VII-I) or (VII-II). Most preferably, the intermediate compound of formula (VII) is a compound of formula (VII-I).

In one embodiment, the intermediate compound of formula (VII) is a compound of formula (VII-II).

The skilled person would appreciate that the compounds of formula (VII) may exist as E and/or Z isomers. Moreover, the individual isomers, may interconvert in solid state, in solution, or under exposure to light. This invention covers processes to prepare all such isomers and mixtures thereof in all proportions. For example a compound of formula (VII-I), (VII-II), (VII-III) or (VII-IV) may be drawn as a compound of formula (VII-Ia), (VII-Ib), (VII-IIa), (VII-IIb), (VII-IIIa), (VII-IIIb), (VII-IVa) or (VII-IVb):

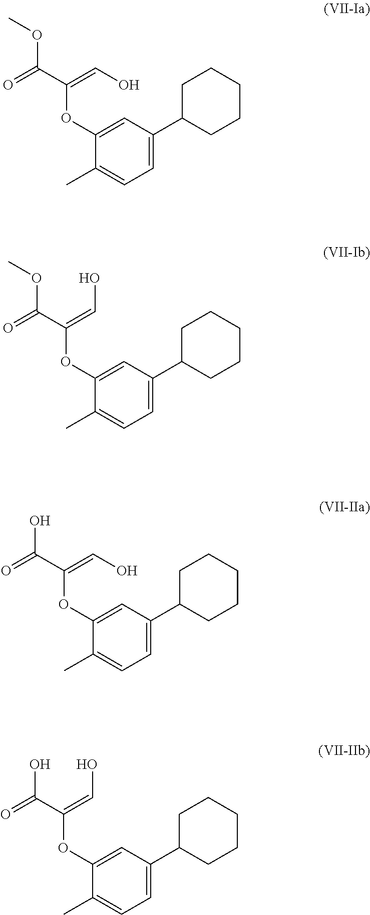 [SMILES: COC(=O)/C(=C/O)Oc1cc(C2CCCCC2)ccc1C ; COC(=O)/C(=C\O)Oc1cc(C2CCCCC2)ccc1C ; Cc1ccc(C2CCCCC2)cc1O/C(=C\O)C(=O)O ; Cc1ccc(C2CCCCC2)cc1O/C(=C/O)C(=O)O]

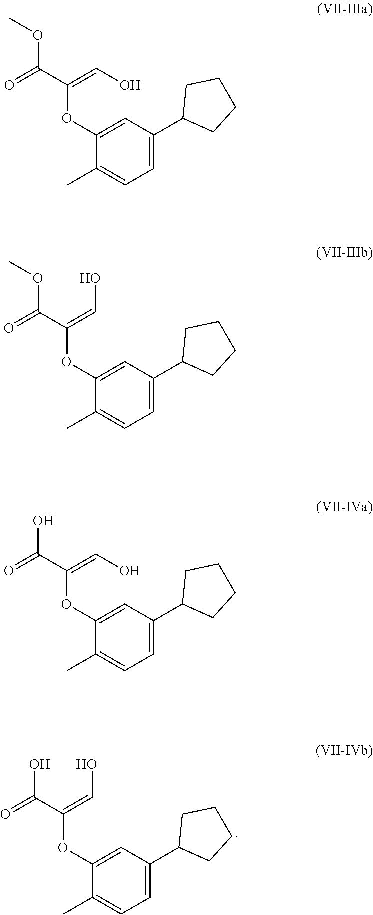 [SMILES: COC(=O)/C(=C/O)Oc1cc(C2CCCC2)ccc1C ; COC(=O)/C(=C\O)Oc1cc(C2CCCC2)ccc1C ; Cc1ccc(C2CCCC2)cc1O/C(=C\O)C(=O)O ; Cc1ccc(C2CCCC2)cc1O/C(=C/O)C(=O)O]

The skilled person would also appreciate that the compounds of formula (VII) may be in equilibrium with alternative tautomeric forms. For example a compound of formula (VII) may be drawn as a compound of formula (VIIa):

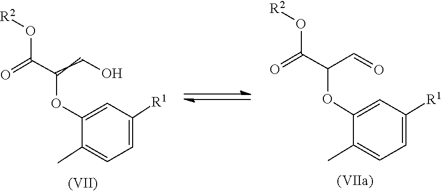

As such the skilled person would appreciate that a compound of formula (VII-I), (VII-II), (VII-III) or (VII-IV) could be drawn as a compound of formula (VI-Ic), (VII-IIc), (VII-IIIc) or (VII-IVc) below:

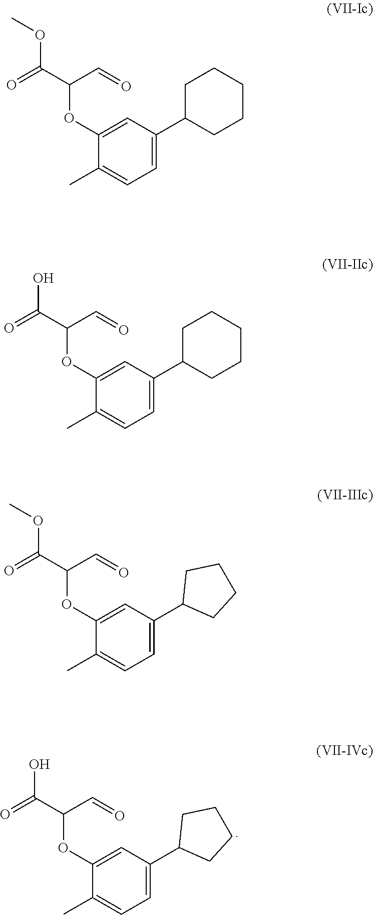 [SMILES: COC(=O)C(C=O)Oc1cc(C2CCCCC2)ccc1C ; Cc1ccc(C2CCCCC2)cc1OC(C=O)C(=O)O ; COC(=O)C(C=O)Oc1cc(C2CCCC2)ccc1C ; Cc1ccc(C2CCCC2)cc1OC(C=O)C(=O)O]

In another embodiment of the invention there is provided an intermediate compound of formula (VIII),

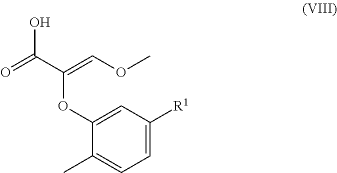

wherein R¹ is as defined herein.

Preferably, the intermediate compound of formula (VIII) is a compound of formula (VIII-I) or (VIII-II) below,

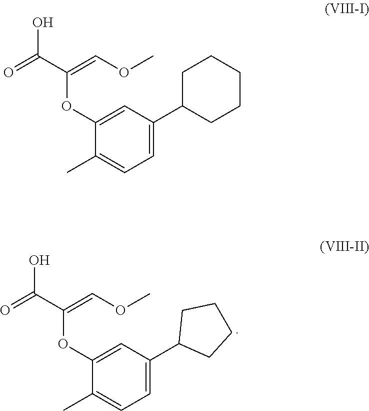 [SMILES: CO/C=C(\Oc1cc(C2CCCCC2)ccc1C)C(=O)O ; CO/C=C(\Oc1cc(C2CCCC2)ccc1C)C(=O)O]

In one embodiment of the invention there is provided the use of a compound of formula (I), (or a salt thereof)

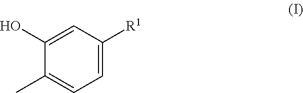

for preparing a compound of formula (VI)

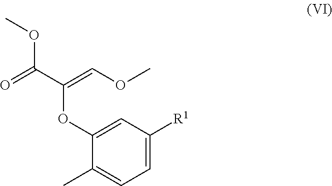

wherein R¹ is as defined herein. Preferably, R¹ is selected from the group consisting of cyclopropyl, cyclobutyl, cyclopentyl and cyclohexyl. More preferably, R¹ is selected from the group consisting of cyclopropyl, cyclopentyl and cyclohexyl. Even more preferably, R¹ is cyclopentyl or cyclohexyl. Most preferably, R¹ is cyclohexyl.

In another embodiment of the invention there is provided the use of a compound of formula (V),

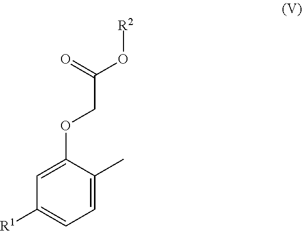

for preparing a compound of formula (VI)

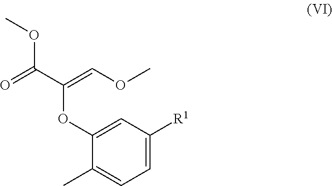

wherein R¹ and R² are as defined herein. Preferably, R¹ is selected from the group consisting of cyclopropyl, cyclobutyl, cyclopentyl and cyclohexyl and R² is hydrogen or methyl. More preferably, R¹ is selected from the group consisting of cyclopropyl, cyclopentyl and cyclohexyl and R² is hydrogen or methyl.

Even more preferably, there is provided the use of a compound selected from the group consisting of a compound of formula (V-I), (V-II), (V-III) and (V-IV) for preparing a compound of formula (VI). Even more preferably still, there is provided the use of a compound of formula (V-I) or (V-II) for preparing a compound of formula (VI). Most preferably, there is provided the use of a compound of formula (V-I) for preparing a compound of formula (VI).

In one embodiment, there is provided the use of a compound of formula (VII) for preparing a compound of formula (VI). Preferably, there is provided the use of a compound selected from the group consisting of a compound of formula (VII-I), (VII-II), (VII-III) and (VII-IV) for preparing a compound of formula (VI). More preferably, there is provided the use of a compound of formula (VII-I) or (VII-II) for preparing a compound of formula (VI). Most preferably, there is provided the use of a compound of formula (VII-I) for preparing a compound of formula (VI).

Compounds of formula (II) (ortho-cresol), (III) and (IV) are either known in the literature or are commercially available.

The present invention further provides a process as referred to above, wherein the compound of formula (I) is further reacted with a compound of formula (IV),

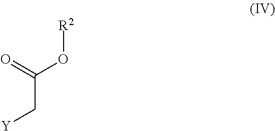

wherein Y is a suitable leaving group (preferably, Y is selected from the group consisting of halogen, CF₃S(O)₂O—, (p-tolyl)S(O)₂O— and CH₃S(O)₂O— more preferably, chloro or bromo, even more preferably, chloro) and R² is selected from the group consisting of hydrogen and C₁-C₆alkyl (preferably R² is hydrogen or methyl, more preferably R² is methyl),
to give a compound of formula (V),

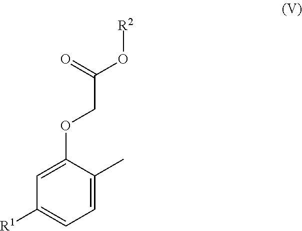

wherein R¹ and R² are as defined herein. Preferably, R¹ is selected from the group consisting of cyclopropyl, cyclobutyl, cyclopentyl and cyclohexyl and R² is selected from the group consisting of hydrogen and C₁-C₆alkyl. More preferably, R¹ is selected from the group consisting of cyclopropyl, cyclopentyl and cyclohexyl and R² is hydrogen or methyl. Even more preferably, R¹ is cyclopentyl or cyclohexyl and R² is hydrogen or methyl. Even more preferably, R¹ is cyclohexyl and R² is hydrogen or methyl. Most preferably, R¹ is cyclohexyl and R² is methyl.

The present invention further provides a process as referred to above, wherein the compound of formula (I) is further converted to a compound of formula (VI)

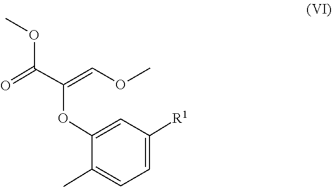

wherein R¹ is as defined herein. Preferably, R¹ is selected from the group consisting of cyclopropyl, cyclobutyl, cyclopentyl and cyclohexyl. More preferably, R¹ is selected from the group consisting of cyclopropyl, cyclopentyl and cyclohexyl. Even more preferably, R¹ is cyclopentyl or cyclohexyl. Most preferably, R¹ is cyclohexyl.

The skilled person would appreciate that the compounds of formula (VI) may exist as E and/or Z isomers. Moreover, the individual isomers, may interconvert in solid state, in solution, or under exposure to light. This invention covers processes to prepare all such isomers and mixtures thereof in all proportions. For example a compound of formula (VI) may be drawn as a compound of formula (VIb):

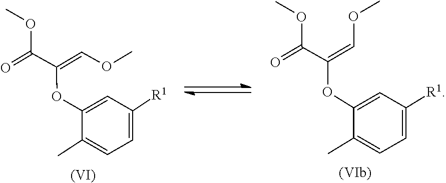

As such the skilled person would appreciate that a compound of formula (VI-I) or (VI-II) could be drawn as a compound of formula (VI-Ib) or (VI-IIb) below:

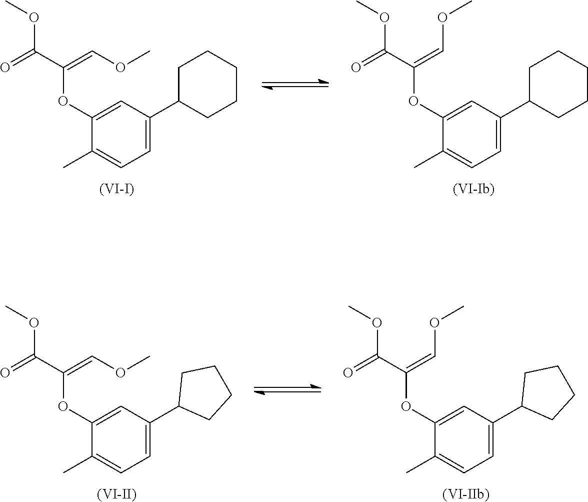

Compounds of formula (VI) are known to have microbiocidal activity, in particular, fungicidal activity, for example, see WO 2020/193387. The compounds of formula (VI) (including a compound of formula (VI-I) or (VI-II)), or fungicidal compositions comprising compounds of formula (VI) (including a compound of formula (VI-I) or (VI-II)) may be useful for combating phytopathogenic fungi (e.g Phakopsora pachyrhizi) containing a mutation in the mitochondrial cytochrome b conferring resistance to Qo inhibitors (e.g strobilurins such as azoxystrobin, pyraclostrobin, picoxystrobin and trifloxystrobin or fenamidone or famoxadone), wherein the mutation is F129L.

The present invention further provides a process as referred to above, wherein the compound of formula (V),

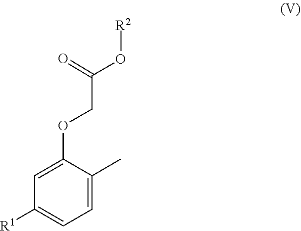

wherein R¹ and R² are as defined herein,
is further converted (for example via formylation and methylation) to a compound of formula (VI)

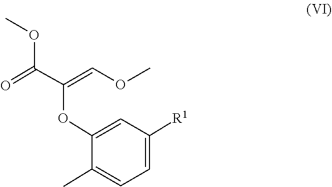

wherein R¹ is as defined herein. Preferably, R¹ is selected from the group consisting of cyclopropyl, cyclobutyl, cyclopentyl and cyclohexyl. More preferably, R¹ is selected from the group consisting of cyclopropyl, cyclopentyl and cyclohexyl. Even more preferably, R¹ is cyclopentyl or cyclohexyl. Most preferably, R¹ is cyclohexyl.

In a preferred embodiment there is provided a process for the preparation of a compound of formula (VI),

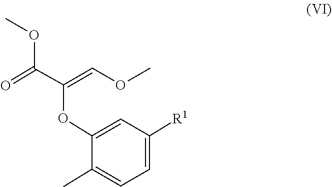

comprising:
(i) reacting a compound of formula (V),

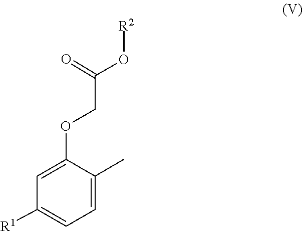

wherein R¹ and R² are as defined herein,
with a formylating agent (preferably methyl formate or trimethyl orthoformate) in the presence of a base (preferably a base selected from the group consisting of sodium methoxide, potassium methoxide, lithium methoxide, cesium methoxide, tetrabutylammonium methoxide, sodium tert-butoxide, potassium tert-butoxide, sodium isopropoxide and potassium isopropoxide, more preferably a base selected from the group consisting of sodium methoxide and potassium methoxide) to give a compound of formula (VII),

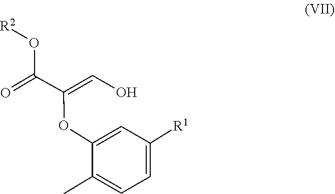

wherein R¹ and R² are as defined herein,
and,
(ii) reacting the compound of formula (VII) with a methylating agent (preferably the methylating agent is methyl iodide or dimethyl sulfate) in the presence of a base (preferably the base is selected from the group consisting of sodium hydroxide, potassium hydroxide, sodium carbonate and potassium carbonate) to give a compound of formula (VI).

In another preferred embodiment there is provided a process for the preparation of a compound of formula (VI),

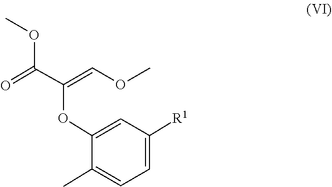

wherein R¹ is as defined herein,
comprising:
(i) reacting a compound of formula (II)

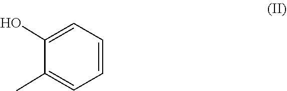

with a compound of formula (III)

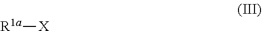

wherein R^{1a} and X are as defined herein,
in the presence of an acid to give a compound of formula (I),

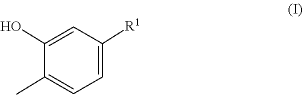

wherein R¹ is as defined herein,
and
(ii) reacting the compound of formula (I) with a compound of formula (IV),

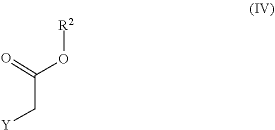

wherein Y and R² are as defined herein,
to give a compound of formula (V),

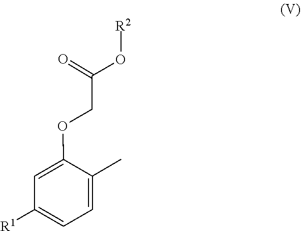

wherein R¹ and R² are as defined herein,
and
(iii) reacting a compound of formula (V) with a formylating agent (preferably methyl formate or trimethyl orthoformate) in the presence of a base (preferably a base selected from the group consisting of sodium methoxide, potassium methoxide, lithium methoxide, cesium methoxide, tetrabutylammonium methoxide, sodium tert-butoxide, potassium tert-butoxide, sodium isopropoxide and potassium isopropoxide, more preferably a base selected from the group consisting of sodium methoxide and potassium methoxide) to give a compound of formula (VII),

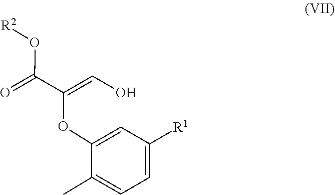

wherein R¹ and R² are as defined herein,
and,
(iv) reacting the compound of formula (VII) with a methylating agent (preferably the methylating agent is methyl iodide or dimethyl sulfate) in the presence of a base (preferably the base is selected from the group consisting of sodium hydroxide, potassium hydroxide, sodium carbonate and potassium carbonate) to give a compound of formula (VI).

Scheme 1 below describes the reactions of the invention in more detail. The substituent definitions are as defined herein.

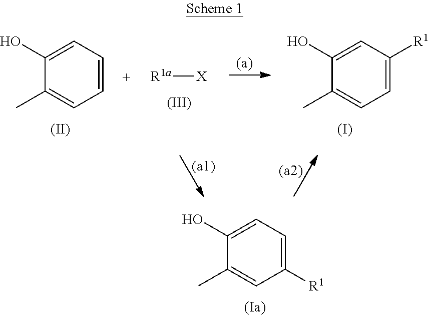

Step (a) Friedel-Crafts Alkylation:

Compounds of formula (I) can be prepared by reacting a compound of formula (II)

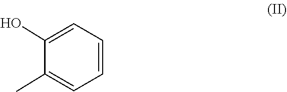

with a compound of formula (III)

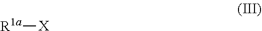

wherein R^{1a} and X are as defined herein;
in the presence of an acid to give a compound of formula (I)

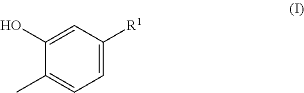

wherein R¹ is as defined herein.

Typically the process described in step (a) is carried out in the presence of a homogeneous or heterogeneous acid including solid or polymer supported acids (such as, but not limited to, zeolites or activated alumina). Preferably, the process described in step (a) is carried out in the presence of a Bronsted acid or a lewis acid, or a mixture of acids, such as but not limited to, trifluoroacetic acid, phosphoric acid (and derivatives thereof such as polyphosphoric acid), hydrochloric acid, sulfuric acid, bismuth(III) trifluoromethanesulfonate, bismuth(III) chloride, lanthanide trifluoromethanesulfonates (including lanthanum(III) trifluoromethanesulfonate, scandium(III) trifluoromethanesulfonate, yttrium(III) trifluoromethanesulfonate), lanthanide chlorides (including lanthanum(III) chloride, scandium(III) chloride, yttrium(III) chloride), aluminium(III) chloride, boron trifluoride, iron(III) chloride, titanium(IV) chloride, zirconium(IV) chloride, zirconium(IV) oxide chloride or trifluoromethanesulfonic acid. Preferably, the process described in step (a) is carried out in the presence of a lewis acid. More preferably, the process described in step (a) is carried out in the presence of a lewis acid selected from the group consisting of aluminium(III) chloride, iron(III) chloride, titanium(IV) chloride, zirconium(IV) chloride and zirconium(IV) oxide chloride. Even more preferably, the process described in step (a) is carried out in the presence of a lewis acid selected from the group consisting of aluminium(III) chloride, titanium(IV) chloride and zirconium(IV) chloride. Most preferably, the process described in step (a) is carried out in the presence of aluminium(III) chloride.

In one embodiment, the process described in step (a) is carried out in the presence of a Bronsted acid (preferably, trifluoromethanesulfonic acid).

In another embodiment, the process described in step (a) is carried out in the presence of aluminium(III) chloride or trifluoromethanesulfonic acid.

Typically the process described in step (a) is carried out in the presence of a catalytic (substoichiometric) or stoichiometric amount (per mole of a compound of formula (III)) of acid. Preferably, the acid is used in an amount of at least 2 molar equivalents per mole of a compound of formula (III). Preferably, the acid is used in an amount of from 3 to 5 molar equivalents per mole of a compound of formula (III).

Typically the process described in step (a) is carried out in the presence of at least 1 molar equivalent of acid per mole of a compound of formula (II). Preferably, the acid is used in an amount of at least 1.1 molar equivalents per mole of a compound of formula (II). More preferably, the acid is used in an amount of from 1.1 to 2 molar equivalents per mole of a compound of formula (II). Even more preferably, the acid is used in an amount of from 1.1 to 1.5 molar equivalents per mole of a compound of formula (II). Even more preferably still, the acid is used in an amount of from 1.2 to 1.3 molar equivalents per mole of a compound of formula (II).

Preferably, in the process described in step (a) the compound of formula (II) is used in an amount of at least 2 molar equivalents per mole of a compound of formula (III). More preferably, the compound of formula (II) is used in an amount of from 3 to 5 molar equivalents per mole of a compound of formula (III).

Preferably, in the process described in step (a) the compound of formula (II) and the amount of acid used is independently at least 2 molar equivalents per mole of a compound of formula (III). More preferably, the compound of formula (II) and the amount of acid used is independently from 3 to 5 molar equivalents per mole of a compound of formula (III).

The process described in step (a) may be carried out as a neat reaction mixture (the skilled person would appreciate that the starting material ortho-cresol (a compound of formula (II)) or the acid may act as a solvent), or in a solvent, or mixture of solvents, such as but not limited to, chlorobenzene, dichloromethane, dichloroethane, dichlorobenzene or hexane. Preferably the process described in step (a) is carried out in a solvent, wherein the solvent is dichloromethane.

This step can be carried out at a temperature of from −20° C. to 150° C., preferably, from −10° C. to 35° C., more preferably from 0° C. to 20° C.

The skilled person would appreciate that the described step (a) may proceed via intermediacy of a compound of formula (Ia), the para regioisomer,

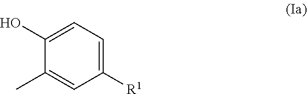

wherein R¹ is as defined herein for compounds of formula (I).

Steps (a1) alkylation and (a2) rearrangement may be carried out in one vessel (one-pot transformation) or sequentially (different reaction vessels).

Typically the process described in step (a2) is carried out in the presence of a homogeneous or heterogeneous acid including solid or polymer supported acids (such as, but not limited to, zeolites or activated alumina). Preferably, the process described in step (a2) is carried out in the presence of Bronsted acid or a lewis acid, or a mixture of acids, such as but not limited to, trifluoroacetic acid, phosphoric acid (and derivatives thereof such as polyphosphoric acid), hydrochloric acid, sulfuric acid, bismuth(III) trifluoromethanesulfonate, bismuth(III) chloride, lanthanide trifluoromethanesulfonates (including lanthanum(III) trifluoromethanesulfonate, scandium(III) trifluoromethanesulfonate, yttrium(III) trifluoromethanesulfonate), lanthanide chlorides (including lanthanum(III) chloride, scandium(III) chloride, yttrium(III) chloride), aluminium(III) chloride, boron trifluoride, iron(III) chloride, titanium(IV) chloride, zirconium(IV) chloride, zirconium(IV) oxide chloride or trifluoromethanesulfonic acid.

The process described in step (a2) may be carried out as a neat reaction mixture (the skilled person would appreciate that the starting material ortho-cresol (a compound of formula (II)) or the acid may act as a solvent), or in a solvent, or mixture of solvents, such as but not limited to, chlorobenzene, dichloromethane, dichloroethane, dichlorobenzene, cyclohexane or hexane.

Step (a2) may be an equilibrium reaction and various methods know to shift the reaction equilibria towards the desired product may be used, including, but not limited to preferential distillation of the desired product, a compound of formula (I) the meta regioisomer.

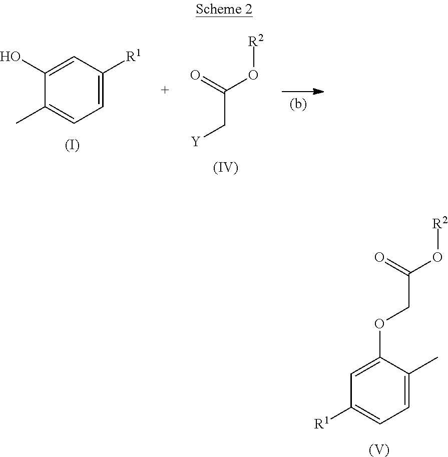

Step (b) alkylation:

Compounds of formula (V) can be prepared by reacting a compound of formula (I)

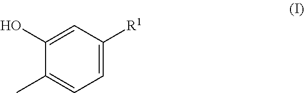

with a compound of formula (IV),

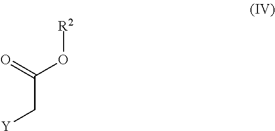

wherein Y is a suitable leaving group (preferably, Y is selected from the group consisting of halogen, CF₃S(O)₂O—, (p-tolyl)S(O)₂O— and CH₃S(O)₂O—, more preferably, chloro or bromo, even more preferably, chloro) and R² is selected from the group consisting of hydrogen and C₁-C₆alkyl (preferably R² is hydrogen or methyl, more preferably R² is methyl),
to give a compound of formula (V),

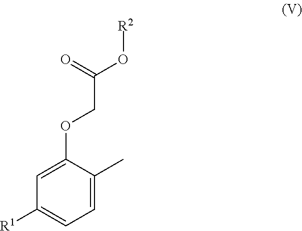

wherein R¹ and R² are as defined herein.

Typically the process described in step (b) can be carried out as a neat reaction mixture, however it may also be carried out in a solvent, or mixture of solvents, such as but not limited to, methanol, ethanol, propanol, isopropanol, tert-butanol, butanol, 3-methyl-1-butanol, tetrahydrofuran, 2-methyltetrahydrofuran, tert-butylmethylether, dimethyl carbonate, toluene, anisole, cumene (isopropylbenzene), p-xylene, o-xylene, m-xylene, xylene iso-mix, mesitylene, chlorobenzene, dichlorobenzene, trifluorobenzene, nitrobenzene, ethylbenzene, dichloromethane, N,N-dimethylformamide, N,N-dimethylacetamide, N-methyl pyrrolidone (NMP), acetonitrile, propionitrile, butyronitrile or benzonitrile (or derivative thereof e.g 1,4-dicyanobenzene). Preferably process step (b) is carried out in acetonitrile, propionitrile or butyronitrile (or mixtures thereof). More preferably, process step (b) is carried out in acetonitrile.

Typically the process described in step (b) can be carried out in the presence of a base or mixture of bases, for example but not limited to, potassium carbonate, sodium carbonate, caesium carbonate, sodium methoxide, potassium methoxide, sodium tert-butoxide, potassium tert-butoxide, potassium hydroxide, sodium hydroxide, trialkyl amines (for example, triethylamine) or amidines (for example, 1,8-diazabicyclo(5.4.0)undec-7-ene). Preferably, process step (b) is carried out in the presence of a base or mixture of bases selected from the group consisting of potassium carbonate, sodium carbonate, caesium carbonate, sodium methoxide, potassium methoxide, sodium tert-butoxide, potassium tert-butoxide, potassium hydroxide and sodium hydroxide. More preferably, process step (b) is carried out in the presence of potassium carbonate or sodium carbonate. Even more preferably, process step (b) is carried out in the presence of potassium carbonate.

The process described in step (b) can be performed in a biphasic system (for example toluene and water) in the presence of a phase transfer catalyst (PTC) such as tetraalkylammonium salt (for example, tetrabutylammonium bisulphate).

Preferably the amount of a compound of formula (IV) used is at least 1 molar equivalent per mole of a compound of formula (I). More preferably, the amount of a compound of formula (IV) used is from 1.05 to 3 molar equivalent per mole of a compound of formula (I).

Typically the process described in step (b) can be carried out at a temperature of from 0° C. to 120° C., preferably, from 10° C. to 50° C.

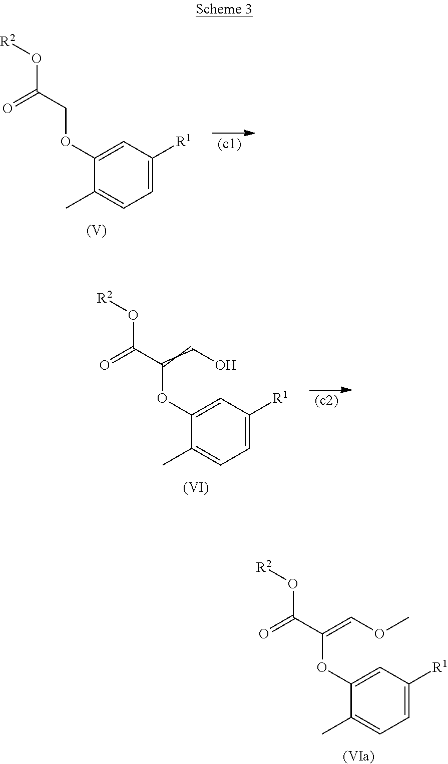

Step (c1):

The process described in step (c1) to convert a compound of formula (V) (wherein R¹ and R² are as defined herein) to a compound of formula (VII) (wherein R¹ and R² are as defined herein) can be carried out in the presence of a base (such as, but not limited to, sodium methoxide, potassium methoxide, lithium methoxide, cesium methoxide, tetrabutylammonium methoxide, sodium tert-butoxide, potassium tert-butoxide, sodium isopropoxide or potassium isopropoxide) and a formylating agent (such as, but not limited to, methyl formate or trimethyl orthoformate). Preferably, the process described in step (c1) is carried out in the presence of a base selected from the group consisting of sodium methoxide, potassium methoxide, lithium methoxide, cesium methoxide and tetrabutylammonium methoxide and methyl formate. More preferably, the process described in step (c1) is carried out in the presence of sodium methoxide and methyl formate.

Alternatively, the process described in step (c1) to convert a compound of formula (V) to a compound of formula (VII) can be carried out via acid promoted beta-hydroxy acrylate formation by treatment with a formylating agent (such as, but not limited to, methyl formate) in the presence of an acid (such as, but not limited to, titanium tetrachloride).

Typically the process described in step (c1) is carried out in the absence of additional solvent or in the presence of a solvent, or mixture of solvents, such as but not limited to, acetic acid, propionic acid, methanol, ethanol, propanol, isopropanol, tert-butanol, butanol, 3-methyl-1-butanol, tetrahydrofuran, 2-methyltetrahydrofuran, diethylether, tert-butylmethylether, tent-amyl methyl ether, cyclopentyl methyl ether, dimethoxymethane, diethoxymethane, dipropoxy methane, 1,3-dioxolane, ethyl acetate, dimethyl carbonate, dichloromethane, dichloroethane, N,N-dimethylformamide, N,N-dimethylacetamide, N-methyl pyrrolidone (NMP), toluene, anisole, cumene (isopropylbenzene), p-xylene, o-xylene, m-xylene, xylene iso-mix, mesitylene, chlorobenzene, dichlorobenzene, trifluorobenzene, nitrobenzene, ethylbenzene, acetonitrile, propionitrile, butyronitrile, benzonitrile (or derivative thereof e.g 1,4-dicyanobenzene), 1,4-dioxane or sulfolane. Preferably the process described in step (c1) is carried out in the absence of additional solvent, or in the presence of a solvent, or mixture of solvents, selected from the group consisting of methanol, ethanol, propanol, isopropanol, tert-butanol, butanol, tetrahydrofuran, 2-methyltetrahydrofuran and toluene. More preferably the process described in step (c1) is carried out in the absence of additional solvent, or in the presence of a solvent, or mixture of solvents, selected from the group consisting of tetrahydrofuran, 2-methyltetrahydrofuran and toluene. Even more preferably the process described in step (c1) is carried out in the presence of a solvent, wherein the solvent is tetrahydrofuran.

Typically the process described in step (c1) can be carried out at a temperature of from −10° C. to 80° C., preferably, from 0° C. to 50° C.
Step (c2):

The process described in step (c2) to convert a compound of formula (VII) (wherein R¹ and R² are as defined herein) to a compound of formula (Via) (wherein R¹ and R² are as defined herein) can be carried out in the presence of a base (such as, but not limited to, sodium hydroxide, potassium hydroxide, sodium carbonate or potassium carbonate) and a methylating agent (such as, but not limited to, methyl iodide or dimethyl sulfate). Preferably, the process described in step (c2) is carried out in the presence of a base selected from the group consisting of sodium hydroxide, potassium hydroxide, sodium carbonate and potassium carbonate and dimethyl sulfate. More preferably, the process described in step (c2) is carried out in the presence of potassium carbonate and dimethyl sulfate.

Typically the process described in step (c2) is carried out in the absence of additional solvent or in the presence of a solvent, or mixture of solvents, such as but not limited to water, toluene, N,N-dimethylformamide, N,N-dimethylacetamide, N-methyl pyrrolidone (NMP), p-xylene, o-xylene, m-xylene, xylene iso-mix, acetonitrile, propionitrile, butyronitrile or benzonitrile (or derivative thereof e.g 1,4-dicyanobenzene). Preferably, the process described in step (c2) is carried out in the absence of additional solvent or in the presence of a solvent, or mixture of solvents, selected from the group consisting of acetonitrile, propionitrile, butyronitrile and benzonitrile. More preferably, process step (c2) is carried out in the presence of a solvent, wherein the solvent is acetonitrile.

The process described in step (c2) can be performed in a biphasic system (for example toluene and water) in the presence of a phase transfer catalyst (PTC) such as tetraalkylammonium salt (for example, tetrabutylammonium bisulphate).

Typically the process described in step (c2) can be carried out at a temperature of from −10° C. to 120° C., preferably, from 0° C. to 50° C.

The skilled person would appreciate that process steps (c1) and (c2) can be carried out in separate process steps, wherein the intermediate compounds can be isolated at each stage. Alternatively, the process steps (c1) and (c2) can be carried out in a one-pot procedure wherein the intermediate compounds produced are not isolated. Thus, it is possible for process steps (c1) and (c2) to be conducted in a batch wise or continuous fashion.

In a preferred embodiment steps (c1) and (c2) are carried out in the same solvent.

The skilled person would also appreciate that for process steps (c1) and (c2), wherein R² is hydrogen an additional alkylation step may be required to prepare compounds of formula (VI),

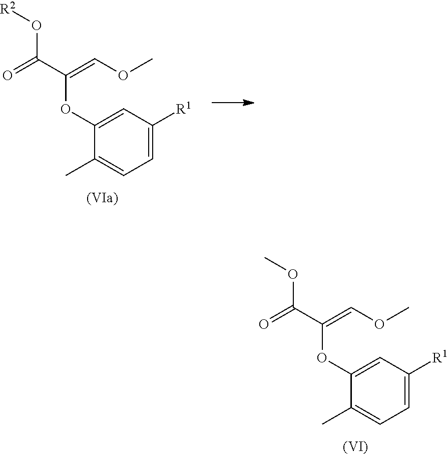

Such an additional step may be carried out in a one-pot procedure (with process steps (c1) and (c2)), for example, by using excess methylating agent in step (c2) or in a separate process step.

The skilled person would also appreciate that the temperature of the process according to the invention can vary in each of steps (a), (b), (c1) and (c2). Furthermore, this variability in temperature may also reflect the choice of solvent used.

Preferably, the process of the present invention is carried out under an inert atmosphere, such as nitrogen or argon.

In a preferred embodiment of the invention there is provided a process for the preparation of a compound of formula (I) or a salt thereof:

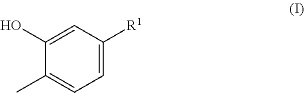

wherein
R¹ is cyclopentyl or cyclohexyl (preferably R¹ is cyclohexyl);
said process comprising:
reacting a compound of formula (II)

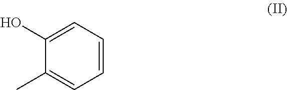

with a compound of formula (III) selected from the group consisting of chlorocyclopentane, chlorocyclohexane, cyclopentanol and cyclohexanol (preferably the compound of formula (III) is chlorocyclohexane or cyclohexanol);
in the presence of an acid (preferably, a lewis acid) to give a compound of formula (I).

Preferably, there is provided a process for the preparation of a compound of formula (I) or a salt thereof:

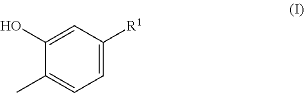

wherein
R¹ is cyclohexyl;
said process comprising:
reacting a compound of formula (II)

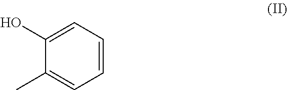

with a compound of formula (III) selected from chlorocyclohexane or cyclohexanol (preferably chlorocyclohexane);
in the presence of a lewis acid selected from the group consisting of aluminium(III) chloride, iron (III) chloride, titanium (IV) chloride and zirconium (IV) chloride (preferably, aluminium(III) chloride) to give a compound of formula (I), wherein the compound of formula (II) and the acid is used independently in an amount of at least 2 molar equivalents (preferably from 3 to 5) per mole of a compound of formula (III).

### EXAMPLES

The following examples further illustrate, but do not limit, the invention. Those skilled in the art will promptly recognise appropriate variations from the procedures both as to reactants and as to reaction conditions and techniques.

The following abbreviations are used: s=singlet; br s=broad singlet; d=doublet; dd=double doublet; dt=double triplet; t=triplet, tt=triple triplet, q=quartet, quin=quintuplet, sept=septet; m=multiplet; GC=gas chromatography, Rₜ=retention time, MH⁺=molecular mass of the molecular cation, M=molar, RT=room temperature.

¹H NMR spectra are recorded at 400 MHz unless indicated otherwise and chemical shifts are recorded in ppm. Samples are measured in CDCl3 as solvent unless indicated otherwise.

### LCMS Methods

Throughout this description, temperatures are given in degrees Celsius and “m.p.” means melting point. LC/MS means Liquid Chromatography Mass Spectroscopy and the description of the apparatus and the methods is as follows:

### Method G

Spectra were recorded on a Mass Spectrometer from Waters (SQD, SQDII Single quadrupole mass spectrometer) equipped with an electrospray source (Polarity: positive and negative ions), Capillary: 3.00 kV, Cone range: 30V, Extractor: 2.00 V, Source Temperature: 150° C., Desolvation Temperature: 350° C., Cone Gas Flow: 50 L/h, Desolvation Gas Flow: 650 L/h, Mass range: 100 to 900 Da) and an Acquity UPLC from Waters: Binary pump, heated column compartment, diode-array detector and ELSD detector. Column: Waters UPLC HSS T3, 1.8 μm, 30×2.1 mm, Temp: 60° C., DAD Wavelength range (nm): 210 to 500, Solvent Gradient: A=water+5% MeOH+0.05% HCOOH, B=Acetonitrile+0.05% HCOOH, gradient: 10-100% B in 2.7 min; Flow (mL/min) 0.85

### Method H

Spectra were recorded on a Mass Spectrometer from Waters Corporation (SQD, SQDII or QDA Single quadrupole mass spectrometer) equipped with an electrospray source (Polarity: positive and negative ions), Capillary: 0.8-3.00 kV, Cone: 5-30 V, Source Temperature: 120-150° C., Desolvation Temperature: 350-600° C., Cone Gas Flow: 50-150 l/h, Desolvation Gas Flow: 650-1000 l/h, Mass range: 110 to 950 Da and an Acquity UPLC from Waters Corporation: Binary pump, heated column compartment, diode-array detector and ELSD. Column: Waters UPLC HSS T3, 1.8 μm, 30×2.1 mm, Temp: 60° C., DAD Wavelength range (nm): 210 to 400, Runtime: 1.5 min; Solvents: A=water+5% MeOH+0.05% HCOOH, B=Acetonitrile+0.05% HCOOH; Flow (ml/min) 0.85, Gradient: 10% B isocratic for 0.2 min, then 10-100% B in 1.0 min, 100% B isocratic for 0.2min, 100-10% B in 0.05min, 10% B isocratic for 0.05 min.

### GCMS Method

GCMS was conducted on a Thermo, MS: ISQ and GC: Trace GC 1310 with a column from Zebron phenomenex: Phase ZB-5ms 15 m, diam: 0.25 mm, 0.25 μm, He flow 1.2 ml/min, temp injector: 250° C., temp detector: 220° C., method: hold 2 min at 40° C., 40° C./min until 320° C., hold 2 min at 320° C., total time 11min.

Cl reagent gas: Methane, flow 1 ml/min.

### Example 1: Preparation of methyl (Z)-2-(5-cyclohexyl-2-methyl-phenoxy)-3-methoxy-prop-2-enoate

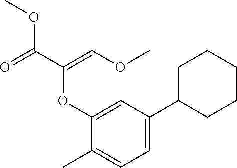 [SMILES: CO/C=C(\Oc1cc(C2CCCCC2)ccc1C)C(=O)OC]

### Step 1: 5-cyclohexyl-2-methyl-phenol

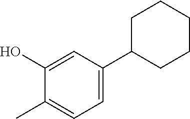

Procedure A: from O-cresol and Chlorocyclohexane:

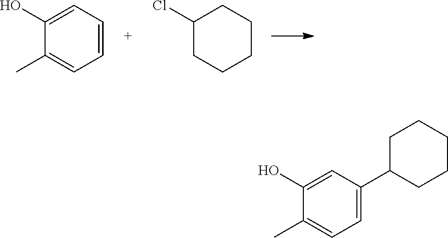

To a solution of o-cresol (27.4 g, 250 mmol, 3.00 equiv.) in dichloromethane (33.4 mL) cooled to 0° C., was added aluminum chloride (36.9 g, 271.3 mmol, 3.25 equiv.) the reaction mixture was stirred at 0° C. for 15 min. then chlorocyclohexane (10.0 mL, 83.5 mmol, 1.00 equiv.) was added dropwise, and after the reaction mixture was stirred at rt for 2h. The resultant reaction mixture was carefully poured into ice-water and extracted with dichloromethane. The total combined organic layer was dried with Na2SO4, filtered, and concentrated in vacuo. The residue was dissolved in tert-butyl methylether and washed three times with 2.0 M aqueous sodium hydroxide solution (70 mL per wash). The organic layer was dried with Na2SO4, filtered, and concentrated in vacuo. The residue was purified by distillation under reduced pressure to give (12.03 g, 58.2 mmol, 70% isolated yield, purity by Q1H NMR: 92%) of 5-cyclohexyl-2-methyl-phenol as a pale-yellow oil.

LC-MS (Method G), Rt=1.13 min, MS: (M+H)=191; 1H NMR (400 MHz, CDCl3) δ ppm: 7.07 (d, 1H), 6.74 (m, 1H), 6.67 (d, 1H), 4.87 (br s, 1H), 2.38-2.50 (m, 1H), 2.25 (s, 3H), 1.83-1.93 (m, 4H), 1.73-1.83 (m, 1 H), 1.33-1.50 (m, 4H), 1.25-1.33 (m, 1H).

Procedure B: from O-cresol and Cyclohexanol:

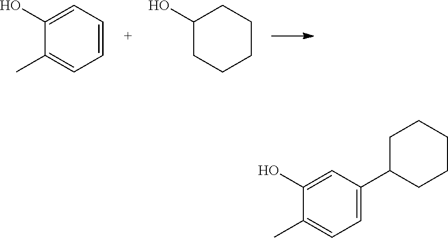

To a solution of o-cresol (0.998 g, 9.13 mmol, 1.05 equiv.) in dichloromethane (8.7 mL) cooled to 0° C., was added aluminum chloride (2.37 g, 17.4 mmol, 2.00 equiv.) the reaction mixture was stirred at 0° C. for 15 min. then cyclohexanol (0.889 g, 8.7 mmol, 1.00 equiv.) was added dropwise, and after the reaction mixture was stirred at rt for 5 h 30 min. The resultant reaction mixture was carefully poured into ice-water and extracted with dichloromethane. The total combined organic layer was dried with Na2SO4, filtered, and concentrated in vacuo. The residue was purified by flash chromatography to give (1.13 g, 4.76 mmol, 55% isolated yield, purity by Q1H NMR: 80%) of 5-cyclohexyl-2-methyl-phenol as a pale-yellow oil.

Procedure C: from O-cresol and Cyclohexene:

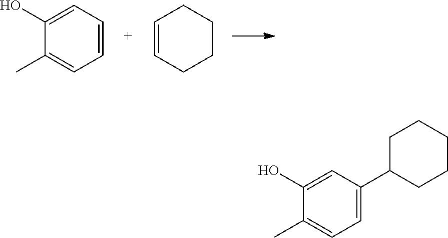

To a solution of o-cresol (3.29 g, 30.1 mmol, 2.50 equiv.) in dichloromethane (6 mL) cooled to 0° C., was added trifluoromethanesulfonic acid (1.83 g, 12.05 mmol, 1.00 equiv.) the reaction mixture was stirred at 0° C. for 15 min. then cyclohexene (1 g, 12.05 mmol, 1.00 equiv.) was added dropwise over 10 min. at 0° C., and after the reaction mixture was stirred at rt for 16 h. The desired product (meta regioisomer) was obtained in the crude reaction mixture.

GC-MS: Rt=7.20 min, MS: (M+H)=191.

### Step 2: methyl 2-(5-cyclohexyl-2-methyl-phenoxy)acetate

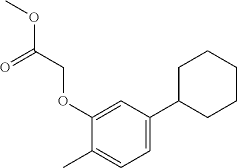 [SMILES: COC(=O)COc1cc(C2CCCCC2)ccc1C]

To a solution of 5-cyclohexyl-2-methyl-phenol (12.0 g, 58.0 mmol, 1 equiv.) in acetonitrile (116 mL) was added potassium carbonate (20.2 g, 145 mmol, 2.50 equiv.) the reaction mixture was heated at 70° C., then methyl chloroacetate (7.89 mL, 9.74 g, 87.0 mmol, 1.50 equiv.) was added dropwise, the reaction mixture was stirred for 4h at 70° C., an excess of methyl chloroacetate (2.63 mL, 3.25 g, 29.0 mmol, 0.5 equiv.) was added and the reaction mixture was stirred for 3h at 80° C. The reaction mixture was filtered, and the filter cake was washed with acetonitrile, the filtrate was concentrated under vacuum, to get a brown oil. This residue was dissolved in methanol and cooled down at 0° C. and the crystallized compound was filtered. The filter cake was washed with cold methanol and dried in vacuo to give 11.9 g, 44.83 mmol, 77.3% isolated yield, purity by Q1H NMR: 99%) of methyl 2-(5-cyclohexyl-2-methyl-phenoxy)acetate as a colorless solid.

LC-MS (Method G), Rt=1.23 min, MS: (M+H)=263; 1H NMR (400 MHz, CDCl3) δ ppm: 7.10 (d, 1H), 6.79 (m, 1H), 6.60 (d, 1H), 4.68 (s, 2H), 3.83 (s, 3H), 2.47 (m, 1H), 2.28 (s, 3H), 1.82-1.92 (m, 4H), 1.73-1.81 (m, 1H), 1.36-1.45 (m, 4H), 1.22-1.32 (m, 1H).

### Step 3: methyl (E/Z)-2-(5-cyclohexyl-2-methyl-phenoxy)-3-hydroxy-prop-2-enoate

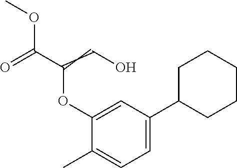 [SMILES: COC(=O)C(=CO)Oc1cc(C2CCCCC2)ccc1C]

To a solution of methyl 2-(5-cyclohexyl-2-methyl-phenoxy)acetate (1 g, 3.81 mmol, 1.00 equiv.) in tetrahydrofuran (3.8 mL) at rt, under argon atmosphere, were added methyl formate (0.584 g, 9.53 mmol, 2.50 equiv.) and sodium methanolate (0.325 g, 5.72 mmol, 1.50 equiv.). The reaction mixture was stirred at rt for 1 h. Ammonium chloride saturated solution in water was added to the reaction mixture which was extracted twice with ethyl acetate. The total combined organic layer was dried with Na2SO4, filtered, and concentrated in vacuo to give methyl 2-(5-cyclohexyl-2-methyl-phenoxy)-3-hydroxy-prop-2-enoate (1.165 g, 3.81 mmol, 100%) as gum which was used directly for the next step.

LC-MS (Method G), Rt=1.09 min, MS: (M+H)=291

### Step 4: methyl (Z)-2-(5-cyclohexyl-2-methyl-phenoxy)-3-methoxy-prop-2-enoate

To a solution of methyl (E/Z)-2-(5-cyclohexyl-2-methyl-phenoxy)-3-hydroxy-prop-2-enoate (1.05 g, 3.62 mmol, 1.00 equiv.) in acetonitrile (7.2 mL) were added potassium carbonate (1.01 g, 7.23 mmol, 2.00 equiv.) and dimethyl sulfate (0.691 g, 5.42 mmol, 1.50 equiv.). The reaction mixture was stirred at rt for 4 h. Ammonium hydroxide solution (25% in water) was added dropwise and the reaction mixture was further stirred at rt for 2 h. The reaction mixture was filtered and the solid was washed with ethyl acetate. The total combined organic layer was dried with Na2SO4, filtered, and concentrated in vacuo to give crude methyl (Z)-2-(5-cyclohexyl-2-methyl-phenoxy)-3-methoxy-prop-2-enoate (1.262 g, 3.48 mmol, 96% isolated yield, purity by Q1H NMR: 84%) as a yellow solid. The crude was recrystallized in cold methanol to give (Z)-2-(5-cyclohexyl-2-methyl-phenoxy)-3-methoxy-prop-2-enoate (0.958 g, 3.17 mmol, 86% isolated yield, purity by Q1H NMR: 99%) as a colourless solid.

LC-MS (Method G), Rₜ=1.21 min, MS: (M+H)=305; 1 H NMR (400 MHz, CDCl3) δ ppm ppm 7.35 (s, 1H), 7.10 (d, 1H), 6.79 (dd, 1H), 6.58 (d, 1H), 3.89 (s, 3H), 3.73 (s, 3H), 2.38-2.47 (m, 1H), 2.34 (s, 3H), 1.80-1.89 (m, 4H), 1.75 (br, 1H), 1.33-1.42 (m, 4H), 1.22-1.32 (m, 1H).

### Preparation of 2-(5-cyclohexyl-2-methyl-phenoxy)acetic acid

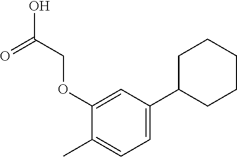 [SMILES: Cc1ccc(C2CCCCC2)cc1OCC(=O)O]

To a solution of methyl 2-(5-cyclohexyl-2-methyl-phenoxy)acetate (0.10 g, 0.36 mmol, 1 equiv.) in methanol (2 mL) was added lithium hydroxide (0.018 g, 0.72 mmol, 2. equiv.) and the reaction mixture was stirred overnight at RT. The contents were then concentrated in vacuo and the resultant crude residue was purified by column chromatography using a cyclohexane/ethyl acetate eluent gradient to afford 0.039 g of 2-(5-cyclohexyl-2-methyl-phenoxy)acetic acid as an off-white solid.

¹H NMR (400 MHz, CDCl₃) δ ppm: 7.09 (d, 1 H), 6.80 (d, 1 H), 6.61 (s, 1H), 4.68 (s, 2H), 2.50-2.40 (m, 1H), 2.26 (s, 3H), 1.89-1.75 (m, 4H), 1.41-1.36 (m, 4H), 1.32-1.22 (m, 2H).

### Example 2: Preparation of methyl (Z)-2-(5-cyclopentyl-2-methyl-phenoxy)-3-methoxy-prop-2-enoate

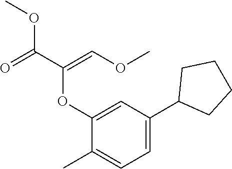 [SMILES: CO/C=C(\Oc1cc(C2CCCC2)ccc1C)C(=O)OC]

### Step 1: Preparation of 5-cyclopentyl-2-methyl-phenol

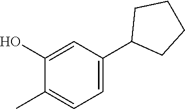

To a solution of o-cresol (3.10 g, 28.4 mmol, 3.00 equiv.) in dichloromethane (9.50 mL) cooled to 0° C., was added aluminum chloride (4.19 g, 30.8 mmol, 3.25 equiv.) and the reaction mixture was stirred at 0° C. for 15 min. Then cyclopentylchloride (1.00 g, 0.99 mL, 9.47 mmol, 1.00 equiv.) was added dropwise and the reaction mixture was stirred at RT for 4h. The reaction mixture was carefully poured into ice-water and extracted with dichloromethane. The residue was dissolved in tert-butyl methylether and washed three times with sodium hydroxide solution (2M) in water. The organic layer was dried with Na2SO4, filtered, and concentrated in vacuo. The residue was purified by flash chromatography to give (1.17 g, 6.62 mmol, 70% isolated yield, purity by Q1H NMR: 98%) of 5-cyclopentyl-2-methyl-phenol as a pale-yellow oil.

LC-MS (Method G), Rt=1.07 min, MS: (M+H)=177; 1H NMR (400 MHz, CDCl3) δ ppm: 7.05 (d, 1H), 20 6.77 (m, 1H), 6.70 (d, 1H), 4.58 (s, 1H), 2.89-3.00 (m, 1H), 2.24 (s, 3H), 2.01-2.11 (m, 2H), 1.76-1.86 (m, 2H), 1.64-1.74 (m, 2H), 1.53-1.63 (m, 2H).

### Step 2: Preparation of methyl 2-(5-cyclopentyl-2-methyl-phenoxy) acetate

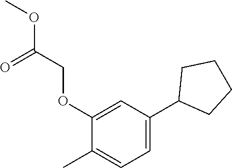 [SMILES: COC(=O)COc1cc(C2CCCC2)ccc1C]

At room temperature, to a solution of 5-cyclopentyl-2-methyl-phenol (300 mg, 1.70 mmol) in acetonitrile (3.40 mL) was added potassium carbonate (594 mg, 4.26 mmol). The resulting pale yellow suspension was heated at 70° C.; then, methyl chloroacetate (0.231 mL, 2.55 mmol) was added dropwise over 1 min. The reaction mixture was stirred at 70° C. for 16h; then, cooled down to room temperature and filtered off. The filter cake was washed with 10 mL of acetonitrile. The filtrate was concentrated to afford the crude title compound as a brown thick oil (chemical yield: 94.5%; purity: 89%). Purification by flash chromatography (Combiflash, silica gel, 0-50% ethyl acetate in cyclohexane) afforded methyl 2-(5-cyclopentyl-2-methyl-phenoxy) acetate as a colourless oil in 84% isolated yield (purity: 99.6%).

¹H NMR (400 MHz, CDCI3) 6 ppm 1.51-1.62 (m, 2 H) 1.65-1.75 (m, 2 H) 1.76-1.88 (m, 2 H) 1.98-2.14 (m, 2 H) 2.89-3.03 (m, 1 H) 3.81-3.87 (m, 3 H) 4.58-4.75 (m, 2 H) 6.06-6.18 (m, 3 H) 6.58-6.68 (m, 1 H) 6.79-6.88 (m, 1 H) 7.02-7.16 (m, 1 H)

LC-MS (Method H): retention time 1.21 min, m/z 249 [M+H⁺].

### Step 3: Preparation of methyl (E/Z)-2-(5-cyclopentyl-2-methyl-phenoxy)-3-hydroxy-prop-2-enoate

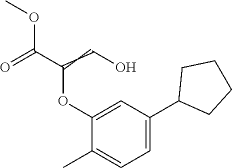 [SMILES: COC(=O)C(=CO)Oc1cc(C2CCCC2)ccc1C]

At room temperature, to a solution of methyl 2-(5-cyclopentyl-2-methyl-phenoxy) acetate (117 mg, 0.471 mmol) in tetrahydrofuran (0.471 mL) under argon was added methyl formate (0.178 mL, 2.83 mmol), followed by sodium methoxide (5.4 M in methanol, 0.170 mL, 0.942 mmol). The resulting pale yellow solution was stirred overnight at room temperature. Water and sat. aq. NH₄Cl were added, and the reaction mixture was extracted twice with ethyl acetate. The organic layer was dried (Na2SO4), filtered and concentrated to afford methyl (E/Z)-2-(5-cyclopentyl-2-methyl-phenoxy)-3-hydroxy-prop-2-enoate as a crude material, which was used in the next step without any purification.

LC-MS (Method H): retention time 1.11 min, m/z 277 [M+H⁺].

### Step 4: Preparation of methyl (Z)-2-(5-cyclopentyl-2-methyl-phenoxy)-3-methoxy-prop-2-enoate

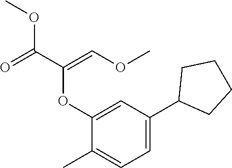 [SMILES: CO/C=C(\Oc1cc(C2CCCC2)ccc1C)C(=O)OC]

At room temperature, to a solution of methyl (E)-2-(5-cyclopentyl-2-methyl-phenoxy)-3-hydroxy-prop-2-enoate (129 mg, 0.467 mmol) in acetonitrile (0.934 mL) was added potassium carbonate (130 mg, 0.934 mmol) under Argon. Then, dimethyl sulfate (0.0671 mL, 0.700 mmol) was added dropwise and the resulting yellow suspension was stirred at room temperature for 1.5 h. Ammonium hydroxide solution (25% in water, 0.120 mL, 0.934 mmol) was added and stirring continued at room temperature for additional 1.5 h before being filtered. The filter cake was washed with ethyl acetate and the filtrate was concentrated to afford the crude title compound as a yellow solid (chemical yield: 56%; purity: 55%). Purification by flash chromatography (Combiflash, silica gel, 0-60% ethyl acetate in cyclohexane) afforded methyl (Z)-2-(5-cyclopentyl-2-methyl-phenoxy)-3-methoxy-prop-2-enoate as a pale yellow solid in 52.5% isolated yield (purity: 90%).

¹H NMR (400 MHz, CDCI3) 6 ppm 1.49-1.58 (m, 2 H) 1.63-1.72 (m, 2 H) 1.74-1.86 (m, 2 H) 1.96-2.10 (m, 2 H) 2.31-2.35 (m, 3 H) 2.86-2.99 (m, 1 H) 3.69-3.76 (m, 3 H) 3.85-3.92 (m, 3 H) 6.58-6.63 (m, 1 H) 6.78-6.84 (m, 1 H) 7.06-7.12 (m, 1 H) 7.30-7.36 (m, 1 H)

LC-MS (Method H): retention time 1.23 min, m/z 291 [M+H⁺].

### Example 3: Preparation of (Z)-2-(5-cyclohexyl-2-methyl-phenoxy)-3-methoxy-prop-2-enoic acid

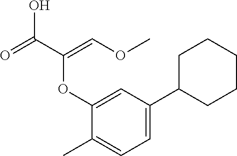 [SMILES: CO/C=C(\Oc1cc(C2CCCCC2)ccc1C)C(=O)O]

To a solution of methyl (Z)-2-(5-cyclohexyl-2-methyl-phenoxy)-3-methoxy-prop-2-enoate (1.2 g, 3.7 mmol) in tetrahydrofuran (11 mL) was added potassium trimethylsilanolate (0.58 g, 4.5 mmol, 1.2 equiv.) portionwise at RT. The reaction mixture was stirred for 14 hour, then diluted with water and acidified with 1N HCl to pH 5. The solution was extracted twice with ethyl acetate and the total combined organic layer was dried over sodium sulfate, filtrated and concentrated under reduced pressure to get a white wax. Purification by preparative reverse phase column chromatography afforded 550 mg (98% pure) of (Z)-2-(5-cyclohexyl-2-methyl-phenoxy)-3-methoxy-prop-2-enoic acid as an off white solid.

LC-MS (Method G), Rₜ=1.07 min, MS: (M+H)=291.

During some reaction sequences to prepare Example 3, reverse phase column chromatography purification afforded a 2-(5-cyclohexyl-2-methyl-phenoxy)-3,3-dimethoxy-propanoic acid by-product which was isolated as a yellow gum:

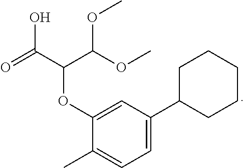 [SMILES: COC(OC)C(Oc1cc(C2CCCCC2)ccc1C)C(=O)O]

1. A process for the preparation of a compound of formula (I) or a salt thereof: 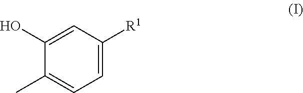
wherein
R¹ is C₃-C₇cycloalkyl;
said process comprising:
reacting a compound of formula (II) 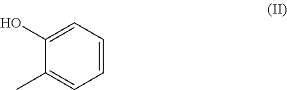
with a compound of formula (III) 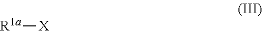
wherein R¹ a is C₃-C₇cycloalkyl and X is halogen or hydroxy; or
R^{1a} is C₃-C₇cycloalkenyl and X is hydrogen;
in the presence of an acid to give a compound of formula (I).

2. A process according to claim 1, wherein R¹ is cyclopentyl or cyclohexyl.

3. A process according to claim 1, wherein a compound of formula (III) is selected from the group consisting of chlorocyclopentane, chlorocyclohexane, cyclopentanol, cyclohexanol, cyclopentene and cyclohexene.

4. A process according to claim 1, wherein R¹ is cyclohexyl and the compound of formula (III) is chlorocyclohexane or cyclohexanol.

5. A process according to claim 1, wherein the acid is a lewis acid.

6. A process according to claim 5, wherein the lewis acid is selected from the group consisting of aluminium(III) chloride, iron(III) chloride, titanium(IV) chloride, zirconium(IV) chloride and zirconium(IV) oxide chloride.

7. A process according to claim 6, wherein the lewis acid is aluminium(III) chloride.

8. A process according to claim 1, wherein the compound of formula (II) is used in an amount of at least 2 molar equivalents per mole of a compound of formula (III).

9. A process according to claim 1, wherein the compound of formula (II) is used in an amount of from 3 to 5 molar equivalents per mole of a compound of formula (III).

10. A process according to claim 1, wherein the acid is used in an amount of at least 1.1 molar equivalents per mole of a compound of formula (II).

11. A process according to claim 1, wherein the compound of formula (I) is further reacted with a compound of formula (IV), 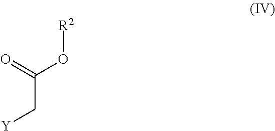
wherein Y is a suitable leaving group and R² is hydrogen or C₁-C₆alkyl;
to give a compound of formula (V), 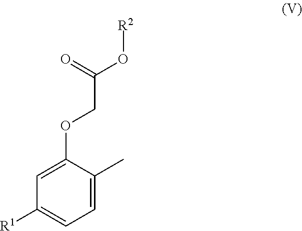
wherein R¹ is as defined in claim 1 and R² is as defined above.

12. A process according to claim 11, wherein Y is chloro.

13. A process according to claim 1, wherein the compound of formula (I) is further converted to a compound of formula (VI) 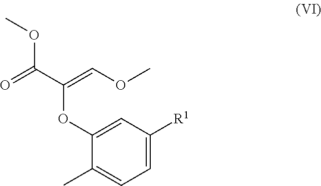
wherein R¹ is as defined in claim 1.

14. A process according to claim 11, wherein the compound of formula (V) is further converted to a compound of formula (VI)

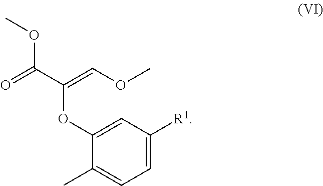

15. A compound selected from the group consisting of a compound of formula (V-I), (V-II), (V-III) and (V-IV) below,

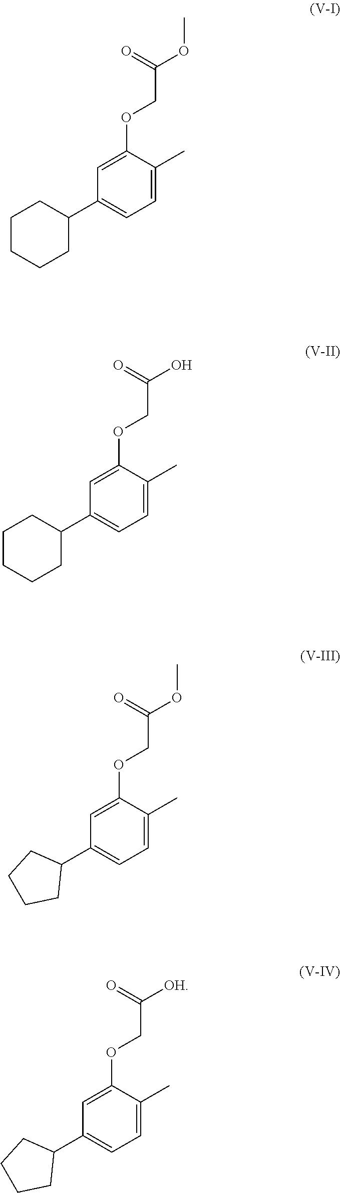 [SMILES: COC(=O)COc1cc(C2CCCCC2)ccc1C ; Cc1ccc(C2CCCCC2)cc1OCC(=O)O ; COC(=O)COc1cc(C2CCCC2)ccc1C ; Cc1ccc(C2CCCC2)cc1OCC(=O)O]

16. Use of a compound of formula (I), 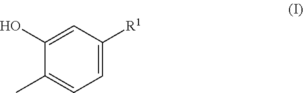
wherein R¹ is as defined in claim 1, for preparing a compound of formula (VI).
